(19) United States
(12) Patent Application Publication
(10) Pub. No.: US 2025/0135101 A1
(43) Pub. Date: May 1, 2025

(54) MEMS MICROPUMP WITH PIEZOELECTRIC ACTIVE VALVE THAT REMAINS CLOSED FOLLOWING MICROPUMP POWER LOSS

(71) Applicant: AITA BIO INC., New York, NY (US)

(72) Inventors: Laurence Heads, Friscp, TX (US); Jacob Menture, New York, NY (US); Dilan Casanovas Mack, New York, NY (US)

(21) Appl. No.: 18/834,008

(22) PCT Filed: Feb. 13, 2023
(86) PCT No.: PCT/US2023/012942

§ 371 Date: Jul. 29, 2024

(51) Int. Cl. A61M 5/142; A61M 5/168

(52) U.S. Cl. CPC A61M 5/14212; A61M 5/16881; A61M 2205/0244; A61M 2205/0294

(57) ABSTRACT

A MEMS device for delivering medicament, the MEMS device configured as a valve for permitting or preventing a flow of the medicament to the user, the MEMS device comprising: a first port and a second port to enable medicament to flow through the MEMS device; first and second wafers that define a cavity that communicates with the first and second ports, the first wafer configured as a membrane, the cavity including a first chamber that communicates with the second port; a valve section including a first piezoelectric actuator layered on the first wafer and configured to cause the membrane to deform and seal the second port, wherein the first piezoelectric actuator is configured to store sufficient charge from an applied voltage to cause the membrane to remain deformed and to maintain the seal on the second port as the MEMS device encounters a loss of power.

### CROSS REFERENCE TO RELATED APPLICATIONS

This application claims priority to U.S. provisional application No. 63/311,441, filed on Feb. 17, 2022 entitled “MEMS Micropump with Piezoelectric Active Valve That Remains Closed Following Micropump Power Loss” which is incorporated by reference herein.

### FIELD OF THE INVENTION

The present invention relates to a MEMS micropump with piezoelectric active valve that remains closed following micropump power loss.

### BACKGROUND OF THE INVENTION

Insulin delivery devices help people with diabetes to conveniently manage their blood sugar. These devices deliver insulin at specific times. Insulin patch pumps or pods are one type of insulin pump. The pods are wearable devices that adhere to the skin of a user using an adhesive patch. The pods incorporate a pump for delivering insulin from through a chamber and internal cannula based on separately acquired CGM sensor readings. It is important to make sure that the these pods deliver the correct amount of insulin times to avoid serious harm to the user. It would be advantageous to provide improvements to these insulin pumps to ensure that only the correct amount of insulin is delivered.

### SUMMARY OF THE INVENTION

A MEMS micropump with piezoelectric active valve that remains closed following micropump power loss is disclosed.

In accordance with an embodiment of the present disclosure, a MEMS device for a device for delivering medicament to a user, the MEMS device configured as a valve for permitting or preventing a flow of the medicament to the user, the MEMS device comprising: a first port and a second port to enable medicament to flow through the MEMS device; first and second wafers that define a cavity therebetween that communicates with the first and second ports, thereby creating a fluid path for a flow of the medicament from the first port to the second port, the first wafer configured as a membrane, the cavity including a first chamber that communicates with the second port; a valve section including a first piezoelectric actuator layered on the first wafer and configured to cause the membrane to deform and seal the second port, thereby preventing fluid flow through the second port, wherein the first piezoelectric actuator is configured to store sufficient charge from an applied voltage from a power source to cause the membrane to remain deformed and to maintain the seal on the second port for a period of time as the MEMS device encounters a loss of power, thereby preventing fluid flow through the second port.

In accordance with yet another embodiment of the disclosure, a device for delivering medicament to a user including a MEMS device configured as a micropump for pumping the medicament into the user and a power source for activating the micropump, the MEMS device comprising: a first port and a second port to enable medicament to flow through the MEMS device; first and second wafers that define a cavity therebetween that communicates with the first and second ports, thereby creating a fluid path for a flow of the medicament from the first port to the second port, the first wafer configured as a membrane that that is adapted to deform into the cavity, the cavity including a first chamber that communicates with the first port and a second chamber that communicates with the first chamber and the second port creating the fluid path and enabling the flow of medicament through the MEMS device; a pump section including a first piezoelectric actuator that is layered on top of the first wafer and is configured to deform the first wafer into the first chamber upon a first applied voltage from the power source to draw into or displace medicament into the first chamber; a first valve section including a second piezoelectric actuator layered on the first wafer and configured to cause the membrane to deform the first wafer into the second chamber and seal the second port, thereby preventing fluid flow through the second port, wherein the second piezoelectric actuator is configured to store sufficient charge from a second applied voltage from the power source to cause the membrane to remain deformed and maintain the seal on the second port for a period of time as the MEMS device encounters a loss of power from the power source, thereby preventing fluid flow through the second port.

In accordance with yet another embodiment of the disclosure, a method of actuating a MEMS device for a device for delivering medicament to a user, the MEMS device configured as a valve for permitting or preventing a flow of the medicament to the user, the MEMS device including a first port and a second port to enable medicament to flow through the MEMS device, first and second wafers that define a cavity therebetween that communicates with the first and second ports, thereby creating a fluid path for a flow of the medicament from the first port to the second port, the first wafer configured as a membrane, the cavity including a first chamber that communicates with the second port, the method comprising: activating a first piezoelectric actuator layered on the first wafer to cause the membrane to deform and seal the second port, thereby preventing fluid flow through the second port, wherein the activating includes applying a voltage V_{c} from a power source to the first piezoelectric actuator to store sufficient charge from the voltage V_{c} to cause the membrane to remain deformed and maintain the seal on the second port for a period of time t as the MEMS device encounters a loss of power, thereby preventing fluid flow through the second port and enabling the user to be notified of the loss of power.

In yet another embodiment of the disclosure, a method of actuating a MEMS device for a device for delivering medicament to a user, the MEMS device configured as a valve for permitting or preventing a flow of the medicament to the user, the MEMS device including a first port and a second port to enable medicament to flow through the MEMS device, first and second wafers that define a cavity therebetween that communicates with the first and second ports, thereby creating a fluid path for a flow of the medicament from the first port to the second port, the first wafer configured as a membrane, the cavity including a first chamber that communicates with the second port, the MEMS device further including a first piezoelectric actuator layered on the first wafer to cause the membrane to deform and seal the second port, thereby preventing fluid flow through the second port, the method comprising: calculating a first voltage to activate the first piezoelectric actuator and to deform membrane and close port; and increasing the first voltage to a second voltage to charge the piezoelectric actuator above the first voltage to the membrane to remain deformed and maintain the seal on the second port for a period of time t as the MEMS device encounters a loss of power, thereby preventing fluid flow through the second port and enabling the user to be notified of the loss of power, wherein increasing the first voltage to the second voltage to ensure the piezoelectric actuator remains deformed for the time t based on the formula: V_{C}(t)=V₀e^{-1/RC}R is the resistance and C the capacitance across the piezoelectric actuator.

### BRIEF DESCRIPTION OF DRAWINGS

FIG. 1 depicts a perspective exploded view of an example micropump.

FIG. 2 depicts a cross sectional view of the micropump in FIG. 1 in fully formed configuration.

FIG. 3 depicts an enlarged sectional view of a valve section shown in FIG. 2.

FIG. 4 depicts a block diagram of example components of device for delivering insulin.

### DETAILED DESCRIPTION OF THE INVENTION

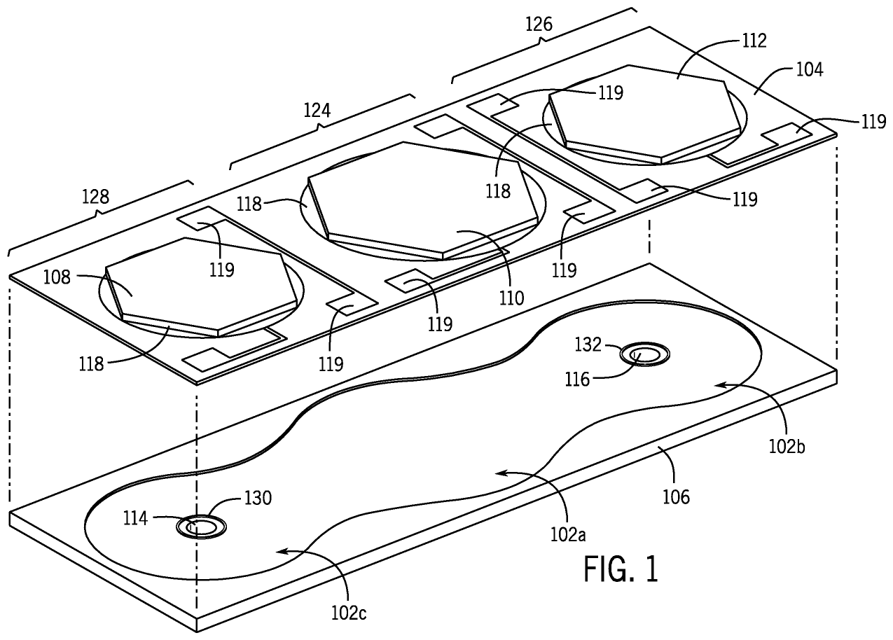

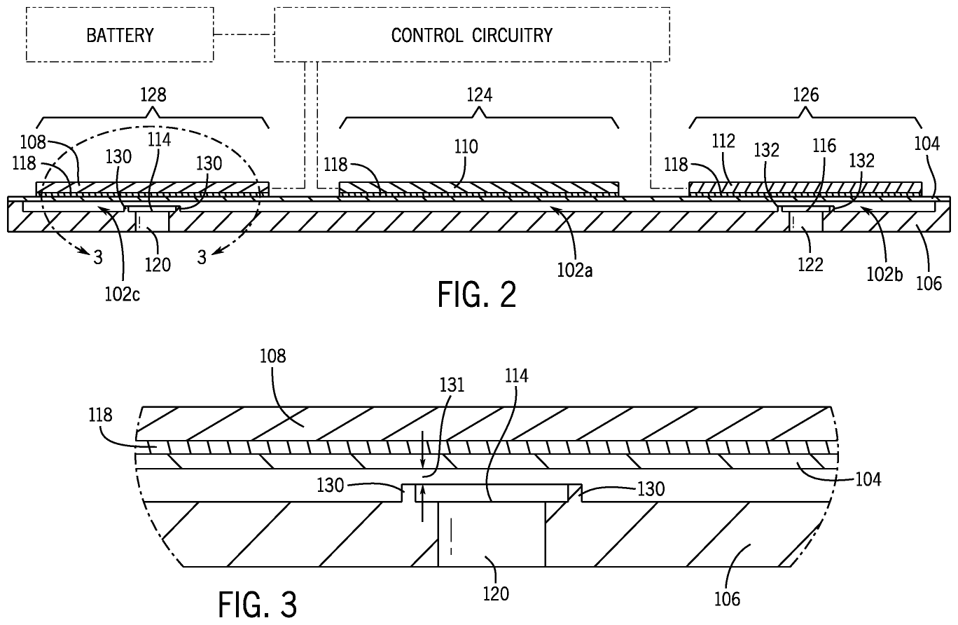

FIG. 1 depicts a perspective exploded view of an example micropump or pump 100. Micropump 100 is part (component) of a device for delivering insulin or other fluid medicament (device 400 described below). The device is configured as a wearable apparatus or system of an infusion system for diabetes management in which continuous glucose monitoring (CGM), insulin delivery and control functionality are provided to ensure insulin is delivered at very precise rates. Device 100 includes several components or modules (not shown) including, among other components, a reservoir for storing the insulin, control circuitry (integrated circuit—C), battery for powering the IC, an insulin needle and a continuous glucose monitoring (CGM) sensor (to name a few components).

Micropump 100 is a MEMS (micro-electro-mechanical systems) device, as known to those skilled in the art, that can be used for pumping fluid, valves used for regulating flow, actuators used for moving or controlling the micropump and valves and/or sensors used for sensing pressure and/or flow. The MEMS device incorporates one or more piezoelectric elements or devices (also known herein as piezoelectric transducers), as known to those skilled in the art. Example piezoelectric devices include piezoelectric actuators and various types of MEMS sensors. As described in more detail below, the piezoelectric devices function as the active element(s) of a pump for pumping fluid and valves for preventing fluid flow and/or a sensor for sensing pressure or flow. (However, various types of MEMS sensors can be used as the sensing elements of the architecture.) Further, other MEMS or non-MEMS structures or technology may also be used to achieve desired results as known to those skilled in the art.) Micropump 100 may be used in a drug infusion system for infusing a drug (i.e., medication) or other fluid to a patient (user). Medication may include small molecule pharmaceutical solutions, large molecule or protein drug solutions, saline solutions, blood or other fluids known to those skilled in the art. Insulin is an example fluid and described below with respect to micropump 100. However, micropump 100 may be used in other environments known to those skilled in the art.

Micropump 100 is configured to maximize micropump efficiency per mm² (i.e., stroke volume per unit area per Watt). To this end, micropump 100 is a cavity substrate that includes cavity 102 with three chambers 102a, 102b and 102c for fluid flow. Micropump 100 incorporates (1) silicon on insulator (SOI) wafer 104 (top wafer) that functions as a membrane for chambers 102a, 102b, 102c. SOI wafer 104 incorporates a buried oxide (BOX) layer and a silicon (Si) layer and (2) double sided polish (DSP) silicon wafer or layer 106 as known to those skilled in the art. (The handle silicon layer of the SOI wafer is removed to form the pump membrane.) SOI wafer 104 sits between silicon wafer 106 and several piezoelectric actuators (transducers) 108, 110, 112 as shown and described below in more detail. A metallization and conductive epoxy layer 118 binds piezoelectric actuators 108, 110 and 112 to SOI wafer 104 as known to those skilled in the art.

In some detail, certain portions of layer 118 underneath corresponding piezoelectric actuators 108, 110, 112 act as ground electrodes while bonding pads 119 function as active electrodes as known to those skilled in the art. Wafer 106 includes inlet and outlet ports 114, 116 that communicate with chambers 102b and 102c of cavity 102 via channels 120, 122, respectively, that extend through the combined wafer structure (wafers 104, 106) as shown. (Note that wafers 106 may alternatively be SOI wafers as known to those skilled in the art.).

Micropump 100 includes pump section 124 and two valve sections 126, 128 that function together to pump fluid through cavity chambers 102a, 102b, 102c of micropump 100. Pump section 124 includes piezoelectric actuator 110 that is layered on top of silicon layer 104 (via metallization layer 118) and it functions to pump or deform/bend silicon layer 104 to draw into or displace liquid contents into cavity chamber 102a from either port 114 or port 116 as desired.

Valve sections 126, 128 are configured as piezoelectric microvalves that function as active valves as described in more detail below. Specifically, valve sections 122, 124 include piezoelectric actuators 108,112 respectively, as well as valve seats 130, 132, respectively. Valve seats 130, 132 are configured to extend into cavity chambers 102b, 102c and to define the introduction of channels 120, 122 and inlet/outlet ports 114, 116. As described above, piezoelectric actuators 108, 112 are layered on top of SOI wafer 104 (via metallization layer 118). Piezoelectric actuators 108, 112 are configured to compress against SOI wafer 104 (membrane) to reach and seal valve seats 130, 132 to thereby discontinue flow through inlet/outlets 114, 116, respectively as needed for proper pump performance, as known to those skilled in the art. (Note that a micropump may include any number of pumps and/or valves as described herein.)

As described above, valve sections 122, 124 are configured as piezoelectric microvalves that function as active valves. In this respect, these valves require activation to be closed in order to prevent free fluid flow which can cause additional drain in power consumption of the system. This is especially important when considering wearable devices. In the example shown in FIG. 1, valve sections 126, 128 are configured to remain physically closed during micropump power loss (e.g., shutoff), and will remain closed for a certain period of time should the electronics in micropump 100 fail. In short, valve section closure is achieved as a function of the piezoelectric actuator material properties (such as PZT) and the actuator's ability to act as a capacitor. As a voltage is applied across the piezoelectric actuators 108,112, membrane 104 will deform down towards valve seats 130, 132 and provide a seal with a high closing pressure.

It is recognized that consideration must be given to the power consumption when valves are held closed in wearable medical devices that employ batteries of limited size and capacity. However, by using the capacitor properties of the piezoelectric actuator materials described herein, membrane deformation may be maintained without increasing power significantly to the device. In this respect, piezoelectric actuators 108, 112 must not be grounded after it is charged. Deformation will remain apart from parasitic loss of charge. This parasitic charge loss must be replenished, i.e., topped up in order to maintain the same closing pressure on valve section 126, 128, but this increase is small as a percentage of the overall original charge provided. The voltage V_{c} (below), and therefore charge across piezoelectric actuators 108,112 decreases exponentially over time and amounts to t seconds after the actuators 108,112 have been charged to voltage V₀. The formula below can be utilized to calculate time constant of decay and therefore the time period “t” as the extent to which it would take for the charge to drop 5%, 10% etc.

$${V}_{C} ( t ) = {V}_{0} {e}^{\frac{- t}{RC}} \qquad ( 1 )$$

R is the resistance and C the capacitance across the piezoelectric actuator.

At this determined time t, the charge would have to be increased, i.e., topped up which allows a true power consumption calculation of the active valve remaining closed over an extended period of time. Using voltage remaining across the piezoelectric actuator after certain time periods, deformation that remains on the actuator may be calculated and the time at which the valve would open determined and therefore provide a time to the user once a failure has been detected to remove micropump 100.

The deflection or deformation of the membrane δ can be calculated based upon the applied voltage V_{d} (V_{C} identified in formula (1) above) on the piezoelectric actuator, the thickness of the membrane t, the area of the membrane A_{d} and the d₃₁ coefficient, (a value that quantifies the deformation amount of a particular piezoelectric actuator when subject to an electric field as known to those skilled in the art), as shown below:

$$δ = \frac{3}{4 π} {d}_{31} \frac{{A}_{d}}{{t}^{2}} {V}_{d} \qquad ( 2 )$$

This allows a time dependent deflection of the membrane to be determined as charge leaks from the piezoelectric actuator (capacitor). So, for example, should the membrane touch the valve seat (lip) (sealing and preventing free flow) at 10V, an overvoltage above 10V is applied so that if power fails in the device, there is reasonable time to alert the user and or to remedy the situation without the membrane returning to an open position that would allow fluid to flow unrestricted to the user. The overvoltage depends on the determined charge leakage constant from the equation (1) above and required time to remain closed. For example, five minutes may be selected before the deformation of the piezoelectric actuator returns to a point whereby it no longer touches the valve lips.

In this respect, voltage on the piezoelectric actuator may be adjusted in order ensure adequate length of time to maintain deformation based on the calculations identified above.

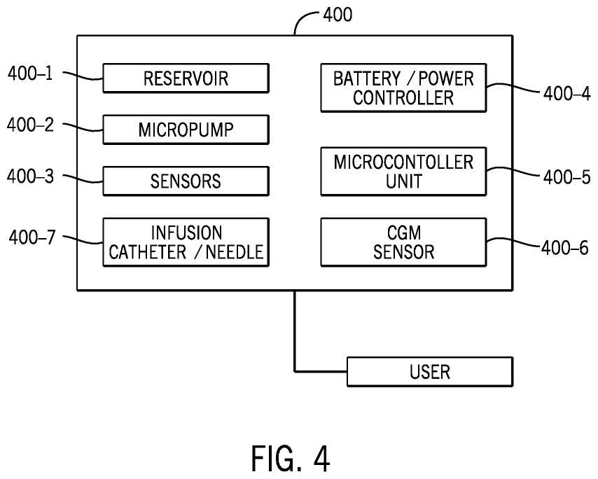

FIG. 4 depicts a block diagram of example components of device 400 for delivering insulin of infusion system as described above. (Device 104 is renumbered as device 400 in FIG. 4.) Specifically, device 400 incorporates several components or modules (not shown) in the fluidic pathway including reservoir 400-1 for storing the insulin, micropump 400-2 (as described hereinabove) for pumping the insulin, sensors 400-3 (e.g., pressure) for sensing various parameters in the system and user and tubing connecting infusion catheter or needle 400-7 to reservoir 400-1. Device 400 also includes microcontroller unit (MCU) 400-4 and battery and power controller 400-5.

Device 400 further also includes CGM sensor 400-6. CGM or continuous glucose monitoring, as known to those skilled in the art, tracks user glucose levels and permits those levels to be used in algorithms that control flow rate. MCU 400-4 controls the operation of micropump 400-2. Infusion needle 400-7 and CGM sensor 400-6 are shown as separate components in FIG. 4 for illustration purposes. Infusion catheter or needle 400-7 and CGM sensor 400-6 may be integrated or may be separate (individually).

Reservoir 400-1 is configured to receive and store insulin for its delivery over a course of about three days, or as needed. However, reservoir size may be configured for storing any quantity of fluid as required.

MCU 400-5 electronically communicates with sensors 400-3 and micropump 400-2 as well as the CGM sensor 400-6, as the monitoring components. Among several functions, MCU 400-5 operates to control the operation of micropump 400-2 to deliver insulin through infusion catheter or infusion needle 400-7 from reservoir 400-1 at specific doses, i.e., flow rates over specified time intervals, based on CGM data converted to desired flow rate via control algorithms.

Battery and power controller 400-4 controls the power to MCU 400-5 and micropump 400-2 to enable those components to function properly as known to those skilled in the art. CGM sensor 400-2 is powered by battery and power controller 400-4 through MCU 400-5.

FIG. 4 depict device 400 with only a few components. Those skilled in the art know that device 400 include additional components.

It shall be understood that this disclosure teaches examples of the illustrative embodiments and that many variations of the invention can easily be devised by those skilled in the art after reading this disclosure and that the scope of the present invention is to be determined by the claims below.

What is claimed is:

1. A MEMS device for a device for delivering medicament to a user, the MEMS device configured as a valve for permitting or preventing a flow of the medicament to the user, the MEMS device comprising:
a first port and a second port to enable medicament to flow through the MEMS device;
first and second wafers that define a cavity therebetween that communicates with the first and second ports, thereby creating a fluid path for a flow of the medicament from the first port to the second port, the first wafer configured as a membrane, the cavity including a first chamber that communicates with the second port;
a valve section including a first piezoelectric actuator layered on the first wafer and configured to cause the membrane to deform and seal the second port, thereby preventing fluid flow through the second port,
wherein the first piezoelectric actuator is configured to store sufficient charge from an applied voltage from a power source to cause the membrane to remain deformed and to maintain the seal on the second port for a period of time as the MEMS device encounters a loss of power, thereby preventing fluid flow through the second port.

2. The device of claim 1 wherein the valve section further includes a valve seat around the second port and extending into the first chamber, the first wafer reaching the valve seat to close the second port.

3. The device of claim 1 wherein the first piezoelectric actuator functions as a capacitor to store the charge from the power source.

4. The device of claim 1 wherein the cavity includes a second chamber in communication with the first chamber and first port and the MEMS device further comprising a pump section including a second piezoelectric actuator that is layered on top of the first wafer and is configured to deform the first wafer into the second chamber upon applied voltage from the power source to draw into or displace medicament into the cavity.

5. The device of claim 1 wherein the medicament is insulin.

6. A device for delivering medicament to a user including a MEMS device configured as a micropump for pumping the medicament into the user and a power source for activating the micropump, the MEMS device comprising:
a first port and a second port to enable medicament to flow through the MEMS device;
first and second wafers that define a cavity therebetween that communicates with the first and second ports, thereby creating a fluid path for a flow of the medicament from the first port to the second port, the first wafer configured as a membrane that that is adapted to deform into the cavity, the cavity including a first chamber that communicates with the first port and a second chamber that communicates with the first chamber and the second port creating the fluid path and enabling the flow of medicament through the MEMS device;
a pump section including a first piezoelectric actuator that is layered on top of the first wafer and is configured to deform the first wafer into the first chamber upon a first applied voltage from the power source to draw into or displace medicament into the first chamber;
a first valve section including a second piezoelectric actuator layered on the first wafer and configured to cause the membrane to deform the first wafer into the second chamber and seal the second port, thereby preventing fluid flow through the second port,
wherein the second piezoelectric actuator is configured to store sufficient charge from a second applied voltage from the power source to cause the membrane to remain deformed and maintain the seal on the second port for a period of time as the MEMS device encounters a loss of power from the power source, thereby preventing fluid flow through the second port.

7. The device of claim 6 wherein the first valve section further includes a valve seat extending around the second port from the second wafer into the second chamber, the first wafer configured to reach the valve seat under the second applied voltage to close the second port, thereby closing the second port.

8. The device of claim 6 wherein the second piezoelectric actuator functions as a capacitor to store the charge from the power source.

9. The device of claim 6 wherein the cavity includes a third chamber in communication with the first chamber and first port and the MEMS device further comprising a second valve section including a third piezoelectric actuator that is layered on top of the first wafer and is configured to deform the first wafer into the third chamber upon a third applied voltage from the power source and seal the first port.

10. The device of claim 9 wherein the second valve section further includes a valve seat extending around the first port from the second wafer into the third chamber, the first wafer configured to reach the valve seat under a third applied voltage from the power source to close the first port, thereby closing the first port.

11. The device of claim 6 wherein the medicament is insulin.

12. A method of actuating a MEMS device for a device for delivering medicament to a user, the MEMS device configured as a valve for permitting or preventing a flow of the medicament to the user, the MEMS device including a first port and a second port to enable medicament to flow through the MEMS device, first and second wafers that define a cavity therebetween that communicates with the first and second ports, thereby creating a fluid path for a flow of the medicament from the first port to the second port, the first wafer configured as a membrane, the cavity including a first chamber that communicates with the second port, the method comprising:
activating a first piezoelectric actuator layered on the first wafer to cause the membrane to deform and seal the second port, thereby preventing fluid flow through the second port,
wherein the activating includes applying a voltage V_{c} from a power source to the first piezoelectric actuator to store sufficient charge from the voltage V_{c} to cause the membrane to remain deformed and maintain the seal on the second port for a period of time t as the MEMS device encounters a loss of power, thereby preventing fluid flow through the second port and enabling the user to be notified of the loss of power.

13. The method of claim 12 further comprising calculating the voltage to charge the first piezoelectric actuator to the voltage V_{c} above a voltage V₀ to ensure the piezoelectric actuator remains deformed for the time t based on the formula:
V_{c}(t)=V₀e^{-t/RC}R is the resistance and C the capacitance across the piezoelectric actuator.

14. A method of actuating a MEMS device for a device for delivering medicament to a user, the MEMS device configured as a valve for permitting or preventing a flow of the medicament to the user, the MEMS device including a first port and a second port to enable medicament to flow through the MEMS device, first and second wafers that define a cavity therebetween that communicates with the first and second ports, thereby creating a fluid path for a flow of the medicament from the first port to the second port, the first wafer configured as a membrane, the cavity including a first chamber that communicates with the second port, the MEMS device further including a first piezoelectric actuator layered on the first wafer to cause the membrane to deform and seal the second port, thereby preventing fluid flow through the second port, the method comprising:
calculating a first voltage to activate the first piezoelectric actuator and to deform membrane and close port;
increasing the first voltage to a second voltage to charge the piezoelectric actuator above the first voltage to the membrane to remain deformed and maintain the seal on the second port for a period of time t as the MEMS device encounters a loss of power, thereby preventing fluid flow through the second port and enabling the user to be notified of the loss of power, wherein increasing the first voltage to the second voltage to ensure the piezoelectric actuator remains deformed for the time t based on the formula: V_{C} (t)=V₀e^{-t/RC} whereby R is the resistance and C is the capacitance across the piezoelectric actuator.

15. The method of claim 14 further comprising calculating deformation for the membrane using the second voltage based on a thickness and area of the membrane.
